(19) United States
(12) Patent Application Publication
(10) Pub. No.: US 2021/0346315 A1
(43) Pub. Date: Nov. 11, 2021

(54) COMPOSITION FOR INHIBITION OR TREATMENT OF BRAIN TUMORS OR SYMPTOMS ATTRIBUTABLE THERETO

(71) Applicant: ENEOS Corporation, Tokyo (JP)

(72) Inventors: Moe KAWAMURA, Tokyo (JP); Masahiro HAYASHI, Tokyo (JP); Yuki KAWASHIMA, Tokyo (JP); Hideaki HARA, Gifu (JP); Shinsuke NAKAMURA, Gifu (JP); Takashi MAOKA, Kyoto (JP)

(73) Assignee: ENEOS Corporation, Tokyo (JP)

(21) Appl. No.: 17/277,706

(22) PCT Filed: Sep. 18, 2019
(86) PCT No.: PCT/JP2019/036538

§ 371 Date: Mar. 18, 2021

(30) Foreign Application Priority Data
Sep. 20, 2018 (JP) 2018-176580
Nov. 5, 2018 (JP) 2018-208402

(51) Int. Cl. A61K 31/122; A61K 31/047; A61P 35/00

(52) U.S. Cl. CPC A61K 31/122; A61P 35/00; A61K 31/047

(57) ABSTRACT

The present invention provides a novel composition for inhibition or treatment of brain tumors or symptoms attributable thereto. More specifically, the present invention provides a composition for inhibition or treatment of brain tumors or symptoms attributable thereto, comprising one or more carotenoids selected from astaxanthin, adonirubin, adonixanthin, zeaxanthin, and a pharmaceutically acceptable salt thereof.

### CROSS-REFERENCE TO RELATED APPLICATIONS

This application is based upon and claims the benefit of priority from Japanese Patent Application No. 2018-176580, filed Sep. 20, 2018, and Japanese Patent Application No. 2018-208402, filed Nov. 5, 2018; the entire contents of which are incorporated by reference.

### TECHNICAL FIELD

The present invention relates to a composition for inhibition or treatment of brain tumors or symptoms attributable thereto.

### BACKGROUND ART

Brain tumor is a general term for tumors occurring in intracranial tissues, and it occurs in not only brain cells but also any intracranial tissues, such as dura mater, arachnoid, and intracranial blood vessels and peripheral nerves. Intracerebral tumors need to be treated so that surrounding normal tissues are not damaged as much as possible. However, many brain tumors infiltrate surrounding brain tissues and spinal cord tissues, and a region where tumor cells and normal brain tissues coexist occurs, and thus it is difficult to remove the brain tumors surgically. Radiation therapy is also likely to damage surrounding normal tissues.

Since glioma, which accounts for about ¼ of brain tumors, also grows while infiltrating surrounding normal brain tissues, total resection by surgery is difficult if the brain functions are attempted to be conserved. Furthermore, radiation therapy and anticancer treatment are often concomitantly used after surgery, but the therapeutic ratio is low.

From these points, there has been needed a composition for inhibition or treatment of brain tumors or symptoms attributable thereto.

Meanwhile, astaxanthin, adonirubin, adonixanthin, and zeaxanthin are one of carotenoids, and they are widely distributed in animals, plant, and microorganisms. Astaxanthin, adonirubin, or adonixanthin have been known to have antianxiety effects (Patent Literature 1).

In addition, Astaxanthin has been reported to have various effects. For example, Patent Literature 2 mentions that a carotenoid mixture containing astaxanthin as a main component can be useful for preventing retinal disorder.

Patent Literature 3 mentions that a lycopene derivative inhibits the proliferation of cells in breast cancer cell systems and prostate cancer cell systems.

However, there has been no report on the relationship between astaxanthin, adonirubin, adonixanthin, and/or zeaxanthin and brain tumors.

### RELATED ART DOCUMENTS

### Patent Literature

### Patent Literature 1: JP 2012-025712 A

### Patent Literature 2: JP 2015-140346 A

### Patent Literature 3: JP 2009-511570 A

### SUMMARY OF THE INVENTION

The present invention provides a novel technical means for effectively inhibiting or treating brain tumors or symptoms attributable thereto.

This time, the present inventors have found that one or more carotenoids selected from astaxanthin, adonirubin, adonixanthin, zeaxanthin, and a pharmaceutically acceptable salt thereof effectively inhibit the growth of brain tumors. The present invention is based on such finding.

The present invention includes the following inventions.

[1] A composition for inhibition or treatment of brain tumors or symptoms attributable thereto, comprising one or more carotenoids selected from astaxanthin, adonirubin, adonixanthin, zeaxanthin, and a pharmaceutically acceptable salt thereof.
[2] The composition according to [1], wherein the carotenoid is a microorganism, animal or plant-derived substance, or a chemical synthetic product.
[3] The composition according to [2], wherein the microorganism is Paracoccus carotinifaciens.
[4] The composition according to any one of [1] to [3], wherein the brain tumors are at least one selected from the group consisting of glioma, meningioma, pituitary adenoma, schwannoma, craniopharyngioma, and metastatic brain tumor.
[5] The composition according to any one of [1] to [4] for intracerebral transfer.
[6] The composition according to any one of [1] to [5] for a human.
[7] The composition according to any one of [1] to [6], which is a food and drink or a food additive.
[8] The composition according to any one of [1] to [7], which is a functional food.
[9] The composition according to any one of [1] to [8], which is a pharmaceutical.
[10] Use of one or more carotenoids selected from astaxanthin, adonirubin, adonixanthin, zeaxanthin, and a pharmaceutically acceptable salt thereof in the production of a composition for inhibition or treatment of brain tumors or symptoms attributable thereto.
[11] A method for inhibiting or treating brain tumors or symptoms attributable thereto in a subject, which includes administering or ingesting an effective dose of one or more carotenoids selected from astaxanthin, adonirubin, adonixanthin, zeaxanthin, and a pharmaceutically acceptable salt thereof to a subject in need thereof.
[12] One or more carotenoids selected from astaxanthin, adonirubin, adonixanthin, zeaxanthin, and a pharmaceutically acceptable salt thereof for inhibition or treatment of brain tumors or symptoms attributable thereto.

According to the present invention, it is possible to effectively inhibit or treat brain tumors or symptoms attributable thereto in a subject. According to the present invention, it is further possible to effectively transfer the carotenoid of the present invention into the brain.

### BRIEF DESCRIPTION OF THE DRAWINGS

FIG. 1 is a graph showing the cell viability after 96 hours of the human-derived glioma cell line U251MG in the control group, the temozolomide group, the astaxanthin group, or the adonixanthin group.

FIG. 2 is a graph showing the cell viability after 96 hours of the mouse-derived glioma cell line GL261 in the control group, the temozolomide group, the astaxanthin group, or the adonixanthin group.

FIG. 3 is a graph showing the cell viability after 72 hours of the human-derived glioma cell line U251MG in the control group, the temozolomide group, or the zeaxanthin group.

FIG. 4 is a graph showing the cell viability after 72 hours of the mouse-derived glioma cell line GL261 in the control group, the temozolomide group, or the adonirubin group.

FIG. 5 is a graph showing the concentration of astaxanthin or adonixanthin in each organ of the brain of the cynomolgus monkey in the astaxanthin-administering monkey or the adonixanthin-administering monkey.

### DETAILED DESCRIPTION OF THE INVENTION

The characteristics of a composition for inhibition or treatment of brain tumors or symptoms attributable thereto of the present invention is that it includes one or more carotenoids selected from astaxanthin, adonirubin, adonixanthin, zeaxanthin, and a pharmaceutically acceptable salt thereof. It is a surprising fact that such carotenoid mentioned above can remarkably inhibit the growth of glioma involved in brain tumors, as shown in Test Examples 1 to 2 mentioned later.

### Carotenoid

The carotenoid in the present invention is one or more selected from astaxanthin, adonirubin, adonixanthin, zeaxanthin, and a pharmaceutically acceptable salt thereof, and preferably adonixanthin or zeaxanthin. Such carotenoid may be, for example, a combination of astaxanthin, adonirubin, and adonixanthin, a combination of astaxanthin, adonixanthin, and zeaxanthin, a combination of astaxanthin, adonirubin, and zeaxanthin, a combination of adonirubin, adonixanthin, and zeaxanthin, a combination of astaxanthin, adonirubin, adonixanthin, and zeaxanthin, a combination of astaxanthin and adonixanthin, a combination of astaxanthin and zeaxanthin, a combination of astaxanthin and adonirubin, a combination of adonirubin and adonixanthin, a combination of adonixanthin and zeaxanthin, or a combination of adonirubin and zeaxanthin. The carotenoid may be a free form or a fatty acid ester form. As the above-mentioned carotenoid, a free form is preferably used in terms of absorbability. The carotenoid may be a stereoisomer such as an optical isomer and a cis-trans isomer. Furthermore, it is preferable to use these carotenoids as an active ingredient.

Astaxanthin is a red pigment and belongs to xanthophyll, which is one of carotenoids. The chemical formula thereof is 3,3′-dihydroxy-β,β-carotene-4,4′-dione (C₄₀H₅₂O₄, molecular weight of 596.852), and the structural formula is represented by the following formula:

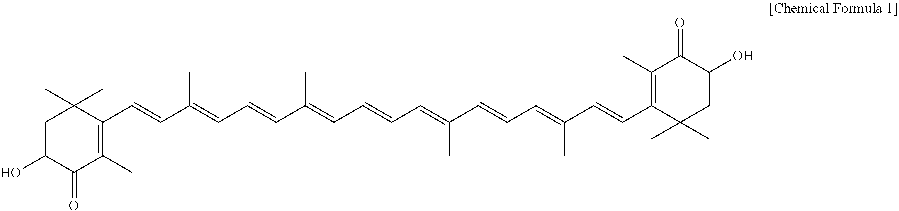 [SMILES: CC1=C(/C=C/C(C)=C/C=C/C(C)=C/C=C/C=C(C)/C=C/C=C(C)/C=C/C2=C(C)C(=O)C(O)CC2(C)C)C(C)(C)CC(O)C1=O]

Examples of an optical isomer of astaxanthin can include at least one selected from the group consisting of a 3S,3'S-isomer, a 3S,3′R-isomer (meso-isomer), and a 3R,3′R-isomer, and the optical isomer is preferably a 3S,3'S-isomer. Astaxanthin may be a cis isomer or a trans isomer of a conjugated double bond in the center of the molecule or a combination thereof. Examples of the cis isomer include a 9-cis isomer, a 13-cis isomer, a 15-cis isomer, a dicis isomer, or a combination thereof. Astaxanthin is preferably a combination of a cis isomer and a trans isomer.

The chemical formula of adonirubin is 3-hydroxy-β,β-carotene-4,4′-dione (C₄₀H₅₂O₃, molecular weight of 580.853), and the structural formula is represented by the following formula:

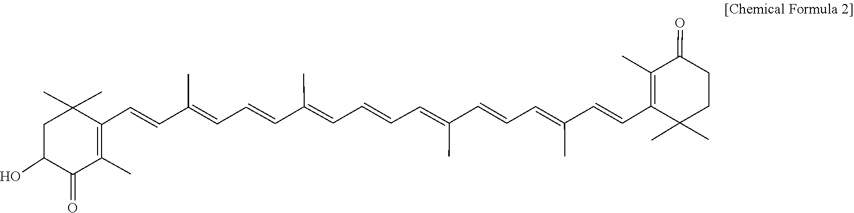 [SMILES: CC1=C(/C=C/C(C)=C/C=C/C(C)=C/C=C/C=C(C)/C=C/C=C(C)/C=C/C2=C(C)C(=O)C(O)CC2(C)C)C(C)(C)CCC1=O]

A cis-trans isomer of adonirubin may be a cis isomer, a trans isomer, or a combination thereof. Examples of the cis isomer can include a 13-cis isomer.

The chemical formula of adonixanthin is 3,3′-dihydroxy-β,β-carotene-4-one (C₄₀H₅₄O₃, molecular weight of 582.869), and the structural formula is represented by the following formula:

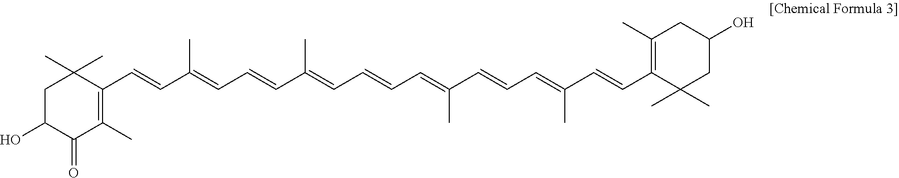 [SMILES: CC1=C(/C=C/C(C)=C/C=C/C(C)=C/C=C/C=C(C)/C=C/C=C(C)/C=C/C2=C(C)C(=O)C(O)CC2(C)C)C(C)(C)CC(O)C1]

Examples of an optical isomer of adonixanthin can include at least one selected from the group consisting of a 3S,3′R-isomer, a 3S,3'S-isomer, a 3R,3'S-isomer, and a 3R,3′R-isomer, and the optical isomer is preferably a 3S,3′R-isomer. A cis-trans isomer of adonixanthin may be a cis isomer, a trans isomer, or a combination thereof. The cis-trans isomer of adonixanthin is preferably a combination of a cis isomer and a trans isomer.

The chemical formula of zeaxanthin is β,β-carotene-3,3′-diol (C₄₀H₅₆O₂, molecular weight of 568.87 to 568.89), and the structure formula is represented by the following formula:

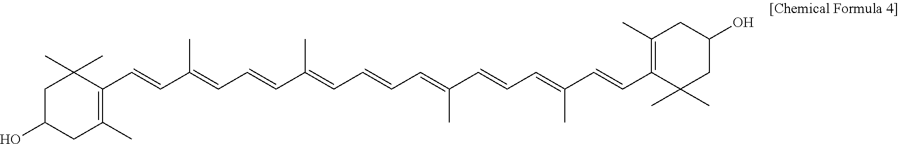 [SMILES: CC1=C(/C=C/C(C)=C/C=C/C(C)=C/C=C/C=C(C)/C=C/C=C(C)/C=C/C2=C(C)CC(O)CC2(C)C)C(C)(C)CC(O)C1]

Examples of an optical isomer of zeaxanthin can include at least one selected from the group consisting of a 3S,3'S-isomer, a 3R,3'S-isomer, and a 3R,3′R-isomer, and the optical isomer is preferably a 3R,3′R-isomer. A cis-trans isomer of zeaxanthin may be a cis isomer, a trans isomer, or a combination thereof. Examples of the cis-trans isomer include an all-trans isomer, a 9-cis isomer, a 13-cis isomer, or a combination thereof. Preferable examples of the stereoisomer include a 3R,3′R-all-trans isomer, a 3R,3′R-9-cis isomer, a 3R,3′R-13-cis isomer, or a combination thereof.

In the present invention, the carotenoid may be in a form of a pharmaceutically acceptable salt, and these salts are also included in the carotenoid in the present invention. In the present invention, the carotenoid may form a salt with an acid or a base. In the present invention, the pharmaceutically acceptable salt is not particularly limited as long as it forms a pharmaceutically acceptable salt with astaxanthin, adonirubin, adonixanthin, and/or zeaxanthin. Specific example thereof include, but are not limited to, hydrohalides (e.g., hydrofluorides, hydrochlorides, hydrobromates, hydroiodides, etc.), inorganic acid salts (e.g., sulfates, nitrates, perchlorates, phosphates, carbonates, bicarbonates, etc.), organic carboxylates (e.g., acetates, oxalates, maleates, tartrates, fumarates, citrates, etc.), organic sulfonates (e.g., methanesulfonates, trifluoromethanesulfonates, ethanesulfonates, benzenesulfonates, toluenesulfonates, camphorsulfonates, etc.), amino acid salts (e.g., aspartates, glutamates, etc.), quaternary amine salts, alkali metal salts (e.g., sodium salts, potassium salts, etc.), and alkaline earth metal salts (e.g., magnesium salts, calcium salts, etc.), etc.

The carotenoid of the present invention may be a commercially available product, or a chemical synthetic product produced by conventional chemical synthesis methods or a microorganism-, animal-, or plant-derived substance (naturally-derived substance) produced by fermentation methods with microorganisms or extraction and purification from microorganisms, animals, or plants or the like can be used. Such microorganism includes bacteria, algae, and yeast. The microorganism-, animal-, or plant-derived substance as used herein is a product obtained from microorganisms, animals, or plants, and the substance may be preferably a Paracoccus microorganism-derived substance, and more preferably a Paracoccus carotinifaciens-derived substance.

Examples of the method for extracting and purifying astaxanthin, adonirubin, and adonixanthin from microorganisms include the following method. Dried bacterial cells of Paracoccus carotinifaciens are subjected to extraction at room temperature using acetone, followed by concentration of the extract with an evaporator. When the concentrated solution is separated into two layers, a hexane-chloroform (1:1) mixture is added to the concentrate to mix well, followed by a separation operation to obtain an organic solvent layer. The organic solvent layer is concentrated to dryness with an evaporator. The concentrated and dried substance is dissolved in chloroform, and each carotenoid is separated with a silica gel column. For example, by further purifying a fraction eluted with acetone:hexane (3:7) using HPLC (Shim-pack PRC-SIL (Shimadzu Corporation), acetone:hexane (3:7)), it is possible to obtain a free form of adonirubin. By concentrating a fraction eluted with acetone:hexane (5:5), followed by allowing to stand at 4° C., it is possible to obtain a free form of astaxanthin as a crystal. Furthermore, by further purifying a fraction eluted with acetone using HPLC (Shim-pack PRC-SIL, acetone:hexane (4:6)), it is possible to obtain a free form of adonixanthin.

Examples of the method for extracting and purifying zeaxanthin from microorganisms include the following method. It is possible to extract zeaxanthin using a water-soluble organic solvent, such as acetone, from a precipitated culture or a precipitated dried substance of Paracoccus microorganism. Furthermore, it is possible to further purify zeaxanthin by performing liquid-liquid extraction after adding a nonpolar organic solvent and/or water to the obtained water-soluble organic solvent extract.

As the method for extracting and purifying zeaxanthin, it is possible to extract and purify zeaxanthin in accordance with the procedure mentioned in US 2014/0113354 A. For example, by extracting the culture with a solvent such as acetone, and by eluting the acetone extract with a silica gel column using an ethyl acetate-hexane (3:7) mixture, it is possible to obtain zeaxanthin.

Furthermore, in the composition of the present invention, a carotenoid mixture containing astaxanthin, adonirubin, adonixanthin, and zeaxanthin, or a carotenoid mixture containing astaxanthin, adonirubin, and adonixanthin may be used. Such carotenoid mixture preferably further contains canthaxanthin, asteroidenone, β-carotene, echinenone, and 3-hydroxyechinenone. For example, the carotenoid mixture extracted from dried bacterial cells of Paracoccus carotinifaciens in accordance with the methods mentioned in JP 2007-261972 A and JP 2009-50237 A contains astaxanthin, adonirubin, and adonixanthin, and preferably further contains at least one selected from the group consisting of canthaxanthin, asteroidenone, β-carotene, echinenone, 3-hydroxyechinenone, and zeaxanthin.

The content of the carotenoid in the composition of the present invention is not particularly limited as long as the effects of the present invention are not impaired, and, for example, is 0.001 to 99% by mass, preferably 0.01 to 99% by mass, more preferably 0.05 to 50% by mass, still more preferably 0.07 to 30% by mass, and further preferably 0.1 to 20% by mass, based on the whole composition. The content of astaxanthin, adonirubin, and adonixanthin in the composition of the present invention can be measured by the HPLC method in accordance with the procedure mentioned in Toxicol Rep. 2014 Aug. 25; 1:582-588. The content of zeaxanthin in the composition of the present invention can be measured by the HPLC method in accordance with the procedure mentioned in [Examples] of JP 6132905 B.

The composition of the present invention can be provided as a composition into which, together with the above-mentioned carotenoids, an orally acceptable or pharmaceutically acceptable additive is formulated if desired. Examples of the additive mentioned above include solvents, solubilizing agents, solubilizers, lubricants, emulsifiers, isotonizing agents, stabilizers, preservatives, antiseptics, surfactants, adjusters, chelating agents, pH adjusters, buffers, excipients, thickeners, coloring agents, aromatics, or perfumes.

The composition of the present invention can be prepared by a known method such as mixing, dissolving, dispersing, and suspending the above-mentioned carotenoid and, if desired, an orally acceptable or pharmaceutically acceptable additive. In preparation of the composition of the present invention, a mixture, a dissolved substance, a dispersed substance, a suspension, etc., prepared by the above-mentioned method may be subjected to homogenization treatment or sterilization treatment, as long as the effects of the present invention are not impaired.

The form of the composition of the present invention is not particularly limited as long as the effects of the present invention are not impaired, and may be solid, semi-solid (including paste and gel), or liquid (including oil and slurry), and the form is preferably solid or liquid.

The dosage form of the composition of the present invention is not particularly limited as long as the effects of the present invention are not impaired, and examples thereof include injection, tablet (e.g., plain tablet, sugar-coated tablet, film-coated tablet, enteric-coated tablet, controlled-release tablet, orally disintegrating tablet, sublingual tablet, chewable tablet, etc.), capsule (e.g., hard capsule, soft capsule), elixir, pill, dust, powder, granule, solution, troche, syrup, dry syrup, emulsion, suspension, liquid, inhalant, aerosol agent, powder inhalant, suppository, ointment, cream, gel, patch, poultice, lotion, drop, ophthalmic ointment, eye drop, and nasal drop. The dosage form of the composition of the present invention is preferably a dosage form for oral intake or administration, and examples thereof include tablet, capsule, pill, dust, powder, granule, syrup, dry syrup, emulsion, liquid, suspension, solution, and troche.

A method for administration or intake of the composition of the present invention is not particularly limited, and examples thereof include injection such as infusion, intravenous injection, intramuscular injection, subcutaneous injection, and intradermal injection, and oral, transmucosal, percutaneous, intranasal, intraoral, etc., administration or intake, and the method is preferably oral intake or administration.

Examples of the composition of the present invention include foods and drinks such as foods or drinks, food additives, feeds, pharmaceuticals, quasi drugs, or cosmetics, and foods and drinks are preferred in terms of simpleness of intake.

The food and drink of the present invention may be one obtained by preparing the composition of the present invention as a food and drink as it is, one into which various proteins, saccharides, fats, trace elements, vitamins, plant extracts, or other active ingredients (e.g., bacteria such as lactic acid bacteria and Bacillus bacteria, fungi such as yeasts, dietary fibers, DHA or EPA) are further formulated, one obtained by making the composition of the present invention into liquid (such as solution), semiliquid, or solid, or one obtained by adding the composition of the present invention to a general food and drink.

Specific examples of the above-mentioned food and drink include instant foods such as instant noodles, pre-packaged foods, canned foods, foods for microwave cooking, instant soups and miso soups, and freeze-dried foods; drinks such as soft drinks, fruit juice drinks, vegetable drinks, soy milk drinks, coffee drinks, tea drinks, powdered drinks, concentrated drinks, alcoholic drinks, and jelly drinks; energy drinks; flour products such as breads, pastas, noodles, cake mixes, and bread crumbs; confectionery such as candies, gummies, jellies, caramels, chewing gums, chocolates, cookies, biscuits, cakes, pies, snacks, crackers, Japanese-style confectionery, and dessert confectionery; nutrition bars; sports bars; seasonings such as sauces, processed tomato seasonings, flavor seasonings, cooking mixes, sauces, dressings, soups, and curry or stew mixes; oils and fats such as processed oils and fats, butter, margarine, and mayonnaise; dairy products such as milk-based drinks, yogurts, lactic acid bacteria drinks, ice creams, and creams; processed agricultural products such as agricultural canned foods, jams and marmalades, and cereals; processed meat foods such as hams, bacons, sausages, and roast pork: and frozen foods, but the food and drink is not limited thereto.

The food and drink of the present invention also includes health foods, supplements, functional foods (e.g., including foods for specified health uses, nutritional functional foods, or foods with function claims), foods for special dietary uses (e.g., including foods for the sick, infant formulas, powdered milk for pregnant and lactating women, or foods for persons with swallowing/chewing difficulties), or liquid modified milk for infants (also referred to as liquid milk for infants). As mentioned later, since the composition of the present invention has an effect of inhibiting or treating brain tumors or symptoms attributable thereto, a food and drink for inhibition or treatment of brain tumors or symptoms attributable thereto is provided. In other words, the food and drink of the present invention can be provided as a food and drink for humans with headache, humans with nausea, humans with visual field defect or double vision, humans with numbness or paralysis in their limbs or face, humans with light-headedness, or humans with slurred speech or impaired speech. Furthermore, a food and drink such as functional foods may be provided with a label of “for persons with headache”, “for persons with nausea”, “for persons with visual field defect or double vision”, “for persons with numbness or paralysis in their limbs or face”, “for persons with light-headedness”, “for persons with slurred speech or impaired speech”, or the like thereon.

The intake or dose of the composition of the present invention is not particularly limited, and can be determined depending on the prescription of the composition, the type of a carotenoid, purity, the type of a subject, age or body weight of a subject, symptoms, the duration of intake or administration, the form of the composition, the method for intake or administration, a combination of a carotenoid other than the carotenoid of the present invention or a drug, and the like. The composition of the present invention is preferably composed of a form of daily intake unit so that the effective dose is for inhibition or treatment of brain tumors or symptoms attributable thereto. For example, when the composition of the present invention is orally taken, one or more carotenoids selected from astaxanthin, adonirubin, adonixanthin, zeaxanthin, and a pharmaceutically acceptable salt thereof can be formulated into the composition so that the intake or dose of the carotenoid is in the range of 0.01 to 10,000 mg, preferably 0.05 to 1,000 mg, and more preferably 0.1 to 100 mg per day per adult with a body weight of 60 kg. The intake or dose of a carotenoid other than the carotenoid of the present invention or a drug used in combination with the carotenoid of the present invention can also be appropriately determined using a clinically used intake or dose of each of them as a standard.

The daily intake or dose of the composition of the present invention is appropriately selected according to the prescription of the composition, etc., like the intake or dose of the composition mentioned above. The daily intake or dose of the composition of the present invention may be, for example, taken by or administered to a subject once or plural times, and is preferably taken by or administered to a subject once. Therefore, the daily number of intake or administration of the composition of the present invention includes 1 to 5 times a day, and is preferably 1 to 3 times a day, and more preferably once a day.

According to one embodiment, a subject to whom the composition of the present invention is applied is not particularly limited as long as the effects of the present invention are not impaired, and is preferably mammals, and more preferably primates such as humans, dogs, and cats. The subject may be healthy subjects (healthy animals) or patients (patient animals).

According to the composition of the present invention, it is possible to inhibit the growth of brain tumors, preferably glioma. Therefore, according to the composition of the present invention, it is possible to inhibit or treat brain tumors or symptoms attributable thereto. Accordingly, according to one embodiment of the present invention, the composition of the present invention is provided as a composition for inhibition or treatment of brain tumors or symptoms attributable thereto. “Inhibition” of a disease or a symptom attributable thereto as used herein includes the meaning of improvement in a disease or a symptom attributable thereto by a non-medical practice, as well as the meaning of “prevention” in which provision is made for expected worsening in advance and occurrence or recurrence of a disease or a symptom attributable thereto is prevented in the bud by a non-medical practice or a medical practice. “Treatment” means improvement in a disease or a symptom attributable thereto by a medical practice. Improvement as used herein includes stopping, alleviating, or delaying the progress or worsening of a disease or a symptom attributable thereto.

The brain tumor mentioned above is not particularly limited, and examples thereof include glioma, meningioma, pituitary adenoma, schwannoma, craniopharyngioma, and metastatic brain tumor, and the brain tumor is preferably glioma. Furthermore, suitable examples of glioma include astrocytoma, oligodendroglioma, glioblastoma, anaplastic astrocytoma, and anaplastic oligodendroglioma. Examples of the symptoms attributable to brain tumors include local symptoms and intracranial hypertension symptoms, examples of the local symptoms include motor paralysis, language disorder, memory disturbance, difficulty in swallowing, personality change, and the like, and examples of the intracranial hypertension symptoms include headache, vomiting, consciousness disorder, visual field defect/visual disorder, and the like.

The composition of the present invention can be transferred into the brain and retained therein. Therefore, according to the other embodiment of the present invention, a composition for intracerebral transfer and/or intracerebral retention is provided. Specific examples of such organ of the brain include cerebrum (e.g., cerebral cortex, cerebral medulla), cerebellum, midbrain, striatum (e.g., striatum putamen, striatum caudate nucleus), hippocampus, medulla oblongata, diencephalon, and the like. Since the composition of the present invention can be transferred into and/or retained in the brain, the composition is advantageous for inhibiting or treating a disease related to the brain or symptoms attributable thereto. Examples of such disease related to the brain include brain tumor (e.g., glioma, meningioma, pituitary adenoma, schwannoma, craniopharyngioma, metastatic brain tumor), cerebral stroke, cerebral infarction, cerebral hemorrhage, subarachnoid hemorrhage, dementia, and the like.

According to the other embodiment of the present invention, there is provided a method for inhibiting or treating brain tumors or symptoms attributable thereto in a subject or a method for transferring the above-mentioned carotenoid into the brain, which includes administering or ingesting a composition including an effective dose of one or more carotenoids selected from astaxanthin, adonirubin, adonixanthin, zeaxanthin, and a pharmaceutically acceptable salt thereof to a subject. According to further the other embodiment of the present invention, there is provided a method for inhibiting or treating brain tumors or symptoms attributable thereto in a subject, which includes administering or ingesting an effective dose of one or more carotenoids selected from astaxanthin, adonirubin, adonixanthin, zeaxanthin, and a pharmaceutically acceptable salt thereof to a subject in need thereof. According to further the other embodiment of the invention, there is provided a method for transferring one or more carotenoids selected from astaxanthin, adonirubin, adonixanthin, zeaxanthin, and a pharmaceutically acceptable salt thereof into the brain, which includes administering or ingesting an effective dose of the above-mentioned carotenoid to a subject in need thereof. “Effective dose” as used herein can be set in the same manner as the content of one or more carotenoids selected from astaxanthin, adonirubin, adonixanthin, zeaxanthin, and a pharmaceutically acceptable salt thereof, etc., in daily intake unit. Examples of the above-mentioned brain tumor include glioma and meningioma, and the brain tumor is preferably glioma. The above-mentioned method can also be applied to a subject only by a non-medical practice. Therefore, according to the other embodiment of the present invention, there is provided a method for inhibiting brain tumors or symptoms attributable thereto in a subject or a method for transferring the above-mentioned carotenoid into the brain (excluding a medical practice, for example, a medical practice for humans), which includes administering or ingesting a composition including an effective dose of one or more carotenoids selected from astaxanthin, adonirubin, adonixanthin, zeaxanthin, and a pharmaceutically acceptable salt thereof to a subject. According to further the other embodiment of the present invention, there is provided a method for inhibiting or treating brain tumors or symptoms attributable thereto in a subject (excluding a medical practice, for example, a medical practice for humans), which includes administering or ingesting an effective dose of one or more carotenoids selected from astaxanthin, adonirubin, adonixanthin, zeaxanthin, and a pharmaceutically acceptable salt thereof to a subject in need thereof. According to further the other embodiment of the invention, there is provided a method for transferring one or more carotenoids selected from astaxanthin, adonirubin, adonixanthin, zeaxanthin, and a pharmaceutically acceptable salt thereof into the brain (excluding a medical practice, for example, a medical practice for humans), which includes administering or ingesting an effective dose of the above-mentioned carotenoid to a subject in need thereof. The above-mentioned method of the present invention can be performed in accordance with the content mentioned herein in the composition of the present invention.

According to the other embodiment of the present invention, there is provided use of one or more carotenoids selected from astaxanthin, adonirubin, adonixanthin, zeaxanthin, and a pharmaceutically acceptable salt thereof for inhibition or treatment of brain tumors or symptoms attributable thereto or for intracerebral transfer of the above-mentioned carotenoid.

According to the other embodiment of the present invention, there is provided use of one or more carotenoids selected from astaxanthin, adonirubin, adonixanthin, zeaxanthin, and a pharmaceutically acceptable salt thereof as a composition for inhibition or treatment of brain tumors or symptoms attributable thereto or for intracerebral transfer of the above-mentioned carotenoid.

According to the other embodiment of the present invention, there is provided use of one or more carotenoids selected from astaxanthin, adonirubin, adonixanthin, zeaxanthin, and a pharmaceutically acceptable salt thereof in the production of a composition for inhibition or treatment of brain tumors or symptoms attributable thereto or for intracerebral transfer of the above-mentioned carotenoid.

According to the other embodiment of the present invention, there is provided one or more carotenoids selected from astaxanthin, adonirubin, adonixanthin, zeaxanthin, and a pharmaceutically acceptable salt thereof for inhibition or treatment of brain tumors or symptoms attributable thereto or for intracerebral transfer of the above-mentioned carotenoid.

Any of the embodiments of the use and the compound (carotenoid) mentioned above can be performed in accordance with the description on the composition or the method of the present invention.

### EXAMPLES

The present invention will be more specifically described below by way of Preparation Examples and Test Examples, but the technical scope of the present invention is not limited to these Examples. Unless otherwise specified, all percentages and ratios used in the present invention are by mass. Unless otherwise specified, the unit and the measurement methods as used herein are in accordance with the JIS Standard.

### Preparation Example 1: Preparation of Adonixanthin, Adonirubin, and Astaxanthin

A free form of astaxanthin, a free form of adonirubin, and a free form of adonixanthin were prepared in accordance with the method mentioned in JP 2012-158569 A. The method will be briefly mentioned below.

Dried bacterial cells of Paracoccus carotinifaciens were subjected to extraction at room temperature using acetone. The extract thus obtained was concentrated with an evaporator, and when the concentrated solution was separated into two layers, a hexane-chloroform (1:1) mixture was added to the concentrate to mix well, followed by a separation operation to obtain an organic solvent layer.

The organic solvent layer thus obtained was concentrated to dryness with an evaporator. The concentrated and dried substance was dissolved in chloroform, and each carotenoid was separated with a silica gel column. Specifically, a fraction eluted with 300 mL of acetone:hexane (3:7) was further purified using HPLC (Shim-pack PRC-SIL (Shimadzu Corporation), acetone:hexane (3:7)) to obtain a free form of adonirubin (hereinafter also simply referred to as adonirubin). A fraction eluted with acetone:hexane (5:5) was concentrated, followed by allowing to stand at 4° C., thus obtaining a free form of astaxanthin as a crystal (hereinafter also simply referred to as astaxanthin). A fraction eluted with acetone was further purified using HPLC (Shim-pack PRC-SIL, acetone:hexane (4:6)) to obtain a free form of adonixanthin (hereinafter also simply referred to as adonixanthin).

### Test Example 1: Investigation of Effects of Astaxanthin or Adonixanthin on Proliferative Capacity of Human- or Mouse-Derived Glioma Cell Line

As the glioma cell lines, the mouse-derived glioma cell line GL261 (transferred from Graduate School of Health Sciences, Gunma University) and the human-derived glioma cell line U251MG (DS Pharma Biomedical Co., Ltd.) were used.

First, the glioma cell lines were seeded in a 96-well plate (FALCON) at 2,000 cells/well. Using Dulbecco's modified Eagle's medium (DMEM) (Nacalai Tesque, Inc.) containing 10% fetal bovine serum (FBS) (Valeant Pharmaceuticals International, Inc.), 100 U/mL penicillin, and 100 μg/mL streptomycin (Meiji Seika Pharma Co., Ltd.), seeded cells were cultured for 24 hours at 37° C. under 5% CO₂. Then, the medium was replaced with DMEM containing 10% FBS so that the concentration was 90 μL/well. After the medium replacement, a test solution was further added according to each test group. Specifically, in the control group, 10 μL of PBS containing 0.1% DMSO (Nacalai Tesque, Inc.) was added (n=6). In the temozolomide group, temozolomide (Tokyo Chemical Industry Co., Ltd.) was dissolved in PBS containing 0.1% DMSO to obtain a temozolomide solution. Then, 10 μL of the temozolomide solution was added so that the final concentration of temozolomide was 300 μM (n=6). In the astaxanthin and adonixanthin groups, astaxanthin or adonixanthin was dissolved in PBS containing 0.1% DMSO to obtain an astaxanthin or adonixanthin solution. Then, 10 μL each of the astaxanthin or adonixanthin solution was added so that the final concentration of astaxanthin or adonixanthin was 0.1, 1.0, 5, or 10 μM (n=6).

The concentration of astaxanthin and adonixanthin was measured by the HPLC method in accordance with the procedure mentioned in Toxicol Rep. 2014 Aug. 25; 1:582-588.

At 72 hours and 96 hours after addition of each test solution, the viable cell count of GL261 and U251MG was measured using cell counting kit-8 (CCK-8) (DOJINDO LABORATORIES). Specifically, using a microplate reader (Varioscan Flash 2.4, Thermo Fisher Scientific K.K.) at the time of addition and 2 hours after addition of CCK-8, the absorbance was measured at 450 nm, and the viable cell count was measured using the absorbance at 650 nm as a reference. For example, in the case of 72-hour treatment, CCK-8 was added 70 hours after addition of the test solution, and the absorbance was measured. The absorbance was measured again 2 hours after addition of CCK-8. The value obtained by subtracting the absorbance immediately after addition of CCK-8 as the background from the absorbance 2 hours after addition of CCK-8 was used as data.

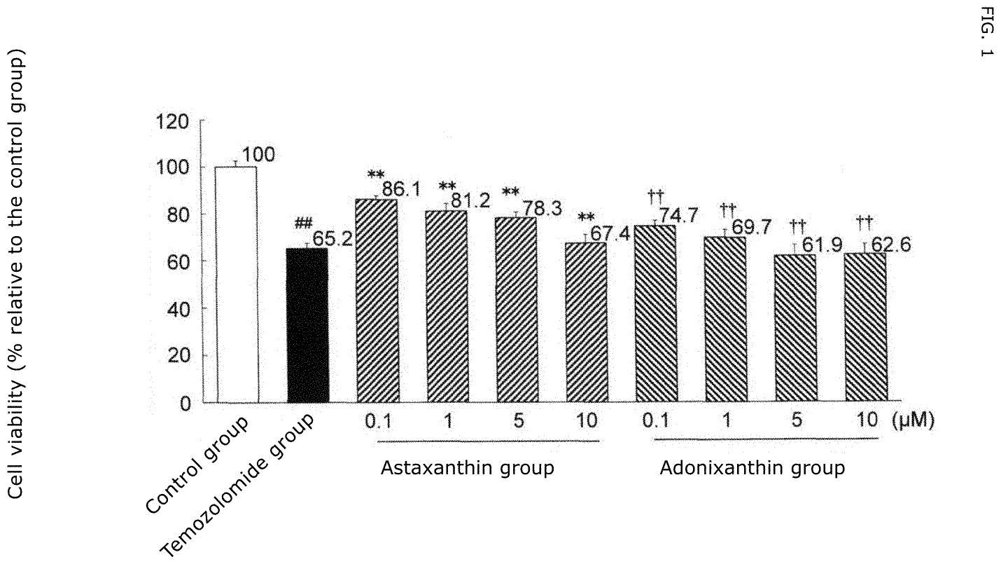

FIG. 1 shows the cell viability after 96 hours of U251MG (proportion (%) of the viable cell count in each group to the viable cell count in the control group). Here, the cell proliferative capacity was evaluated based on the cell viability. The measured value was expressed as mean and standard error. As shown in FIG. 1, in the astaxanthin group and the adonixanthin group, the proliferation was significantly inhibited at any concentration compared with the control group (##: p<0.01 vs the control group (Student's t-test), **: p<0.01 vs the control group (Dunnett's test), ††: p<0.01 vs the control group (Dunnett's test)). Particularly, in the adonixanthin group, the proliferation was inhibited at a higher level compared with the astaxanthin group.

As a result of evaluation of the cell proliferative capacity after 72 hours of U251MG, it was found that the proliferation tends to be concentration-dependently inhibited in the astaxanthin group and the adonixanthin group compared with the control group. Particularly, in the 1, 5, and 10 μM adonixanthin groups, the proliferation is significantly inhibited compared with the control group, showing a proliferation inhibitory effect at a high level.

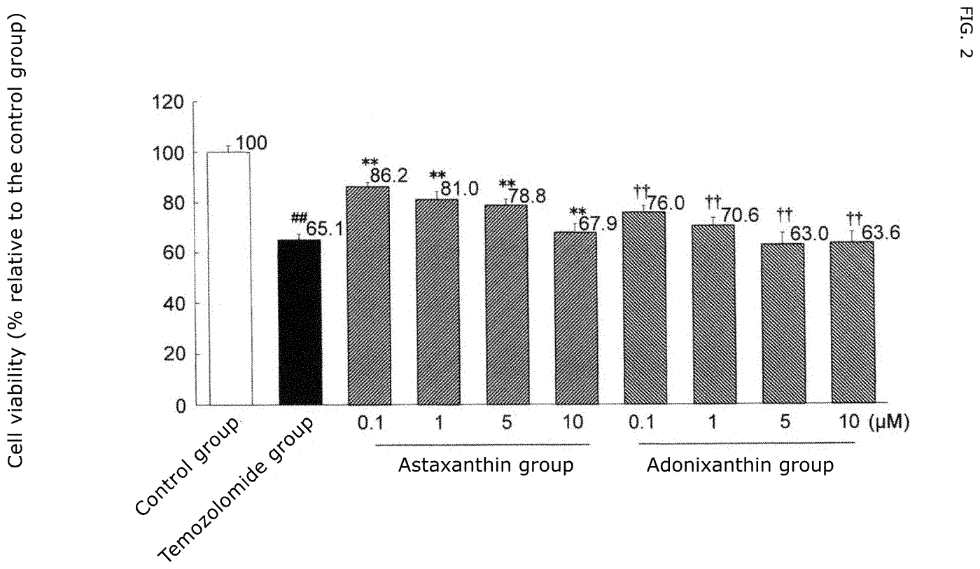

FIG. 2 shows the cell viability after 96 hours of GL261. The measured value was expressed as mean and standard error. As shown in FIG. 2, in the astaxanthin group and the adonixanthin group, the proliferation was significantly inhibited at any concentration compared with the control group (##: p<0.01 vs the control group (Student's t-test), **: p<0.01 vs the control group (Dunnett's test), ††: p<0.01 vs the control group (Dunnett's test)). Particularly, in the adonixanthin group, the proliferation was inhibited at a higher level compared with the astaxanthin group.

As a result of evaluation of the cell proliferative capacity after 72 hours of GL261, it was found that the average value of cell viability tends to concentration-dependently decrease in the astaxanthin group and the adonixanthin group. Particularly, in the 10 μM astaxanthin group and all concentration adonixanthin groups, the proliferation is significantly inhibited compared with the control group, showing a proliferation inhibitory effect at a high level.

### Test Example 2: Investigation of Effects of Adonirubin or Zeaxanthin on Cell Proliferative Capacity of Human- or Mouse-Derived Glioma Cell Line

In the same manner as in Test Example 1, effects of adonirubin or zeaxanthin on the cell proliferative capacity after 72 hours of a human- or mouse-derived glioma cell line were investigated. As adonirubin, the adonirubin obtained in Preparation Example 1 was used, and as zeaxanthin, Zeaxanthin (manufacturer code ASB-00026504, ChromaDex Corporation) was used.

Specifically, the glioma cell lines were seeded in a 96-well plate at 2,000 cells/well. Using DMEM containing 10% FBS, 100 U/mL penicillin, and 100 μg/mL streptomycin, seeded cells were cultured for 24 hours at 37° C. under 5% CO₂. Then, the medium was replaced with DMEM containing 10% FBS so that the concentration was 90 μL/well. After the medium replacement, a test solution was further added according to each test group. Specifically, in the control group, 10 μL of PBS containing 0.1% DMSO (Nacalai Tesque, Inc.) was added (n=6). In the temozolomide group, temozolomide was dissolved in PBS containing 0.1% DMSO to obtain a temozolomide solution. Then, 10 μL of the temozolomide solution was added so that the final concentration of temozolomide was 300 μM (n=6). In the adonirubin and zeaxanthin groups, adonirubin or zeaxanthin was dissolved in PBS containing 0.1% DMSO to obtain an adonirubin or zeaxanthin solution. Then, 10 μL each of the adonirubin or zeaxanthin solution was added so that the final concentration of adonirubin or zeaxanthin was 0.1, 1.0, 5, or 10 μM (n=6).

The concentration of adonirubin was measured by the HPLC method in accordance with the procedure mentioned in Toxicol Rep. 2014 Aug. 25; 1:582-588. The concentration of zeaxanthin was measured by the HPLC method in accordance with the procedure mentioned in [Examples] of JP 6132905 B.

At 72 hours after addition of each test solution, the cell proliferative capacity of GL261 and U251MG was evaluated using CCK-8 in the same manner as in Test Example 1.

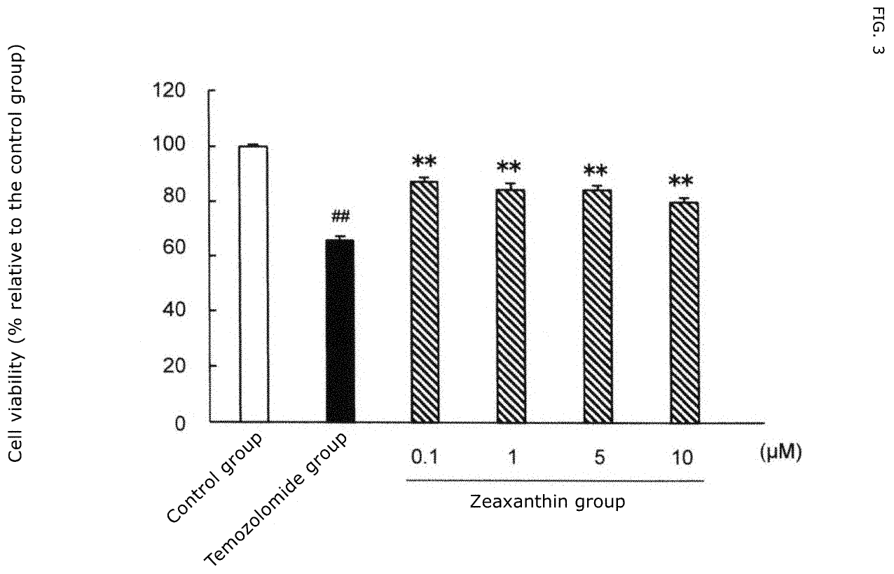

FIG. 3 shows the cell viability after 72 hours of U251MG. The measured value was expressed as mean and standard error. As shown in FIG. 3, in the zeaxanthin group, the proliferation was significantly inhibited at any concentration compared with the control group (##: p<0.01 vs the control group (Student's t-test): **: p<0.01 vs the control group (Dunnett's test)).

In the adonirubin group, it was found that the proliferation tends to be inhibited at any concentration compared with the control group.

Particularly, in the zeaxanthin group, the proliferation was inhibited at a higher level compared with the adonirubin group.

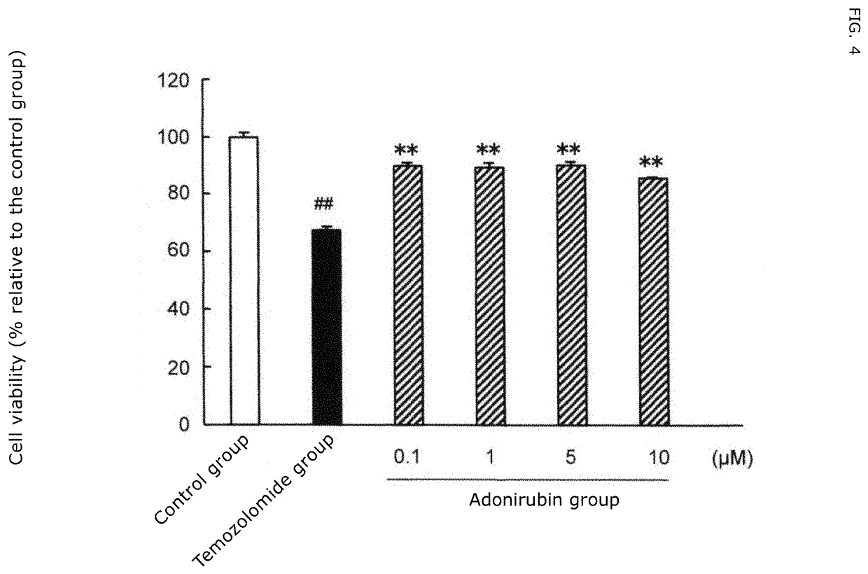

FIG. 4 shows the cell viability after 72 hours of GL261. The measured value was expressed as mean and standard error. As shown in FIG. 4, in the adonirubin group, the proliferation was significantly inhibited at any concentration compared with the control group (##: p<0.01 vs the control group (Student's t-test): **: p<0.01 vs the control group (Dunnett's test)).

In the zeaxanthin group, it was found that the proliferation tends to be inhibited at any concentration compared with the control group. Particularly, in the 10 μM zeaxanthin group, the proliferation is significantly inhibited compared with the control group, showing a proliferation inhibitory effect at a high level.

Furthermore, in the adonirubin group, the proliferation was inhibited at a higher level compared with the zeaxanthin group.

As shown in FIGS. 1 to 4, 10 μM astaxanthin, adonirubin, adonixanthin and zeaxanthin (the present invention 10 μM groups) are 1/30 of the concentration of temozolomide, which is an existing drug. However, when the cell viability in the temozolomide group is defined as 1, the cell viability in the present invention 10 μM groups is about 0.96 to 1.27, showing an almost equivalent cell proliferation inhibitory effect to that in the temozolomide group. From this result, it was found that astaxanthin, adonirubin, adonixanthin, and zeaxanthin can remarkably inhibit the growth of glioma compared with temozolomide, which is an existing drug.

### Preparation Example 2: Preparation of Astaxanthin-Containing Composition (Dosing Solution) and Adonixanthin-Containing Composition (Dosing Solution)

Each of the astaxanthin and the adonixanthin obtained in Preparation Example 1 was weighed, followed by suspension by adding olive oil (product number 150-00276, manufactured by Wako Pure Chemical Industries, Ltd.) to prepare so that each concentration was 10 mg/mL, thus obtaining an astaxanthin dosing solution and an adonixanthin dosing solution. Each dosing solution was prepared at the time of use, and stored on ice and protected from light until administration.

### Test Example 3: Confirmation of Intracerebral Distribution of Astaxanthin or Adonixanthin in Cynomolgus Monkey

As experimental animals, cynomolgus monkeys were used. Two cynomolgus monkeys were used, and the astaxanthin dosing solution was administered to one monkey (astaxanthin-administering monkey), and the adonixanthin dosing solution was administered to the other monkey (adonixanthin-administering monkey). As the dosing solution, the dosing solution obtained in Preparation Example 2 was used. The dosing solution was administered at a dose of 50 mg/kg body weight as astaxanthin or adonixanthin once daily for 10 days (the day of initiation of administration of the dosing solution is defined as day 1). As the administration method, a disposable catheter was inserted from the nasal cavity into the stomach, and the dosing solution was injected into the stomach using a syringe. When the dosing solution was collected into the syringe, the dosing solution was collected while being stirred with a stirrer. The dose in each administration was calculated based on the latest body weight at each administration time point (body weight was measured using an electric balance (HP-40K or GP-40K, both of which were manufactured by A&D Company, Limited) on each of the day of initiation of acclimation, the day of end of acclimation, the day of initiation of administration, and before administration on the day 8 of administration). The administration time was 8:30 to 13:30.

During the administration period of the dosing solution, each cynomolgus monkey was fed about 108 g (about 12 g×9 pieces) of pellets once daily at 14:00 to 16:00, and feed remained by the feeding on the next day (regarding the administration day, before administration) was collected. Each cynomolgus monkey fed tap water ad libitum and maintained on a 12-hour light/dark cycle, at 23±3° C. and with a relative humidity of 50±20%.

Blood was collected 4 hours after the last administration of the dosing solution, and then an aqueous solution (64.8 mg/mL) of pentobarbital sodium (Tokyo Chemical Industry Co., Ltd.) was administered into the cephalic vein at a dose of 0.4 mL/kg to perform anesthesia. After body weight was measured, each monkey was euthanized by exsanguination, and the cerebral cortex, cerebral medulla, cerebellum, midbrain, striatum putamen, striatum caudate nucleus, hippocampus, medulla oblongata, and diencephalon were collected. Each organ thus collected was immediately frozen with liquid nitrogen and stored in a deep freezer (−70° C. or less).

Each of the concentration of adonixanthin in each organ collected from the adonixanthin-administering monkey (concentration relative to the weight of each organ) and the concentration of astaxanthin in each organ collected from the astaxanthin-administering monkey (concentration relative to the weight of each organ) was measured. Specifically, each organ was homogenized, and extraction was repeated with acetone until no color occurred. Then, filtration through a filter was performed to evaporate acetone, and diethyl ether: hexane (2:8, v/v) was added to the solution to extract a carotenoid. Furthermore, evaporation to dryness was performed, and the residue was dissolved in acetone:hexane (2:8, v/v) and subjected to HPLC. As the HPLC device, a Hitachi L-6000 intelligent pump and an L-4250 UV-VIS detector were used. The measurement wavelength was 450 nm, and a column of 5 μm Cosmosil 55L-II (inner diameter of 250×4.6 mm) (Nacalai Tesque, Inc., Japan) was used. As the mobile phase, acetone:hexane (2:8, v/v) was used, and measurement was performed at a flow rate of 1.0 mL/min.

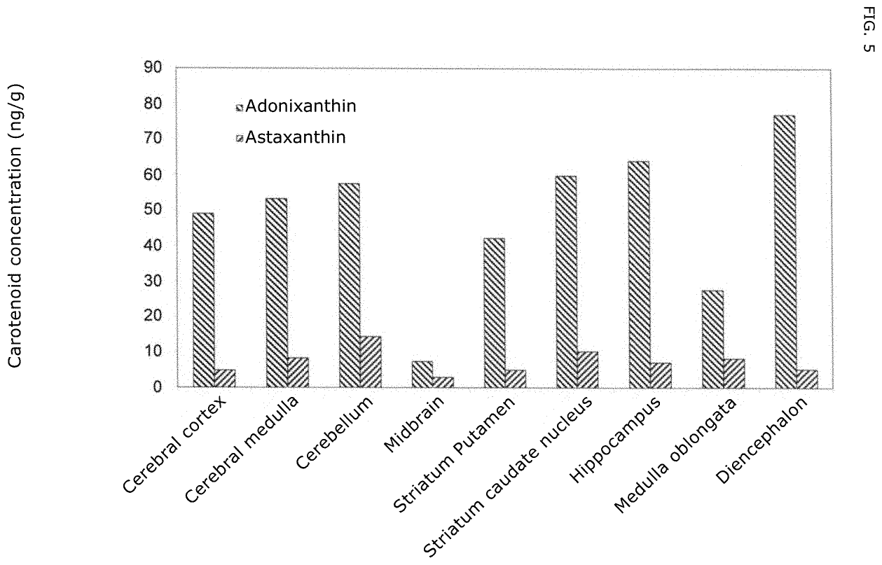

FIG. 5 shows the concentration of astaxanthin and adonixanthin in each organ. As shown in FIG. 5, adonixanthin and astaxanthin were transferred into the brain at a high concentration to retain therein. Particularly, adonixanthin was transferred into the brain at a higher concentration to retain therein, compared with astaxanthin.

1.-10. (canceled)

11. A method for inhibiting or treating brain tumors or symptoms attributable thereto in a subject, which comprises administering or ingesting an effective dose of one or more carotenoids selected from astaxanthin, adonirubin, adonixanthin, zeaxanthin, and a pharmaceutically acceptable salt thereof to a subject in need thereof.

12. (canceled)

13. The method according to claim 11, wherein the carotenoid is a microorganism, animal or plant-derived substance, or a chemical synthetic product.

14. The method according to claim 13, wherein the microorganism is Paracoccus carotinifaciens.

15. The method according to claim 11, wherein the brain tumors are at least one selected from the group consisting of glioma, meningioma, pituitary adenoma, schwannoma, craniopharyngioma, and metastatic brain tumor.

16. The method according to claim 11, wherein the subject is a human.

17. The method according to claim 11, wherein the carotenoid is in the form of a food and drink or a food additive.

18. The method according to claim 11, wherein the carotenoid is in the form of a functional food.

19. The method according to claim 11, wherein the carotenoid is in the form of a pharmaceutical.

20. The method according to claim 14, wherein the brain tumors are at least one selected from the group consisting of glioma, meningioma, pituitary adenoma, schwannoma, craniopharyngioma, and metastatic brain tumor.

21. The method according to claim 20, wherein the subject is a human.

22. The method according to claim 21, wherein the carotenoid is in the form of a food and drink or a food additive.

23. The method according to claim 21, wherein the carotenoid is in the form of a functional food.

24. The method according to claim 21, wherein the carotenoid is in the form of a pharmaceutical.
